(19) United States
(12) Patent Application Publication
(10) Pub. No.: US 2004/0171958 A1
(43) Pub. Date: Sep. 2, 2004

(54) Atrial fibrillation detection via a ventricular lead

(76) Inventors: Stephanie M. Fitts, Middletown, NY (US); H. Toby Markowitz, Roseville, MN (US); Rahul Mehra, Stillwater, MN (US); Michael R.S. Hill, Minneapolis, MN (US); Mark L. Brown, North Oaks, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/358,799

(22) Filed: Feb. 5, 2003

(51) Int. Cl. A61B 5/0402

(52) U.S. Cl. 600/518; 607/14

(57) ABSTRACT

Techniques for detecting atrial fibrillation via a ventricular lead are described. An implantable medical device according to the invention may monitor a ventricular depolarization rate via the ventricular lead in order to detect the effect of conducted atrial fibrillation of the ventricular rate. For example, the implantable medical device may detect an increase in the ventricular rate, or a decrease in the stability of the ventricular rate. The implantable medical device may also detect the effect of delivery of a pacing pulse on the length of an R-R interval subsequent to the delivery. A compensatory pause following delivery of a pacing pulse may indicate the presence of conducted atrial fibrillation. The implantable medical device may store information relating to detected episodes of atrial fibrillation for later review by a physician, so that atrial fibrillation may be diagnosed in situations where it might not otherwise be detected.

### TECHNICAL FIELD

The invention relates to implantable medical devices, and more specifically to detection of atrial fibrillation via an implantable medical device.

### BACKGROUND

Atrial fibrillation is characterized by rapid, irregular, uncoordinated depolarizations of the atria of a heart. These fibrillation depolarizations may originate from an ectopic focus within or proximate to the atria, or may be caused by re-entrant loops. Atrial fibrillation may cause minor pumping inefficiency of the heart, but is generally not immediately life threatening. Indeed, in some cases, an individual may not even notice atrial fibrillation.

Atrial fibrillation may induce rapid and irregular ventricular rhythms. In these cases, irregular atrial fibrillation depolarizations are received by the atrioventricular (“AV”) node and are conducted to ventricles. This phenomenon is referred to as conducted atrial fibrillation. Unlike ventricular fibrillation, atrial fibrillation that is conducted to the ventricles is not immediately life threatening because the atrial fibrillation depolarizations are conducted to the ventricles at much less than a one-to-one ratio. Conducted atrial defibrillation may compromise the pumping efficiency of the heart more drastically than atrial fibrillation in the absence of conduction, and may cause an individual to experience symptoms such as fatigue. However, these symptoms are often not interpreted by the individual as a serious health issue.

Although atrial fibrillation with or without conduction to the ventricles is not immediately life threatening, atrial fibrillation may have potentially life threatening consequences in the long term. For example, atrial fibrillation may cause blood to pool in the left atrium, which, over time, may lead to emboli formation and stroke. Consequently, individuals with diagnosed atrial fibrillation may take anticoagulants to reduce the likelihood of emboli formation and stroke. Unfortunately, because the effects of atrial fibrillation may not be perceived as a serious health issue by individuals experiencing atrial fibrillation, the first diagnosis atrial fibrillation all too often occurs after an individual has already had a stroke.

### SUMMARY

In general, the invention is directed to techniques for detecting atrial fibrillation via a ventricular lead. An implantable medical device according to the invention may monitor a ventricular depolarization rate via the ventricular lead in order to detect the effect of conducted atrial fibrillation on the ventricular rate. The implantable medical device may store information relating to detected episodes of atrial fibrillation for later review by a physician, so that atrial fibrillation may be diagnosed in situations where it might not otherwise be detected. In some embodiments, the implantable medical device may alert the patient of prolonged episodes of atrial fibrillation via an audible alarm, so that the patient may promptly seek medical attention.

In order to detect atrial fibrillation, the implantable medical device may detect an increase in the ventricular rate, or a decrease in the stability of the ventricular rate. The implantable medical device may also detect the effect of delivery of a pacing pulse on the length of an R-R interval subsequent to the delivery. A compensatory pause following delivery of a pacing pulse may indicate the presence of conducted atrial fibrillation.

In one embodiment, the invention is directed to a method in which atrial fibrillation is detected via a ventricular lead. Information relating to the detected atrial fibrillation is stored in a memory.

In another embodiment, the invention is directed to an implantable medical device that includes a memory for storing information and a processor. The processor detects atrial fibrillation via a ventricular lead and stores information relating to the detected atrial fibrillation in the memory.

In another embodiment, the invention is directed to a computer-readable medium containing instructions. The instructions cause a programmable processor to detect atrial fibrillation via a ventricular lead, and store information relating to the detected atrial fibrillation in a memory.

The invention may provide advantages. For example, by monitoring the ventricular rate to detect atrial fibrillation and storing information relating to detected atrial fibrillations, an implantable medical device according to the invention may allow a physician to later review the information and diagnose atrial fibrillation in situations where atrial fibrillation may have otherwise been unsuspected and undetected, such as in situations where the previous condition of the patient had only indicated the need for a simple implantable medical device, such as a single-lead pacemaker operating in VVI mode. This, in turn, would allow the physician to prescribe proper therapy for atrial fibrillation, such as anticoagulation therapy to reduce the risk of stroke resulting from atrial fibrillation.

The details of one or more embodiments of the invention are set forth in the accompanying drawings and the description below. Other features and aspects of the invention will be apparent from the description and drawings, and from the claims.

### BRIEF DESCRIPTION OF DRAWINGS

FIG. 1 is perspective diagram illustrating an example implantable medical device for detecting atrial fibrillation via a ventricular lead.

FIG. 2 is a block diagram further illustrating the implantable medical device of FIG. 1.

FIG. 3 is a timing diagram used to illustrate exemplary methods for detecting atrial fibrillation via a ventricular lead.

FIG. 4 is a flowchart illustrating an example method for detecting atrial fibrillation via a ventricular lead.

FIG. 5 is a flowchart illustrating another example method for detecting atrial fibrillation via a ventricular lead.

FIG. 6 is a flowchart illustrating another example method for detecting atrial fibrillation via a ventricular lead.

FIG. 7 is a timing diagram used to illustrate an exemplary method for detecting or confirming atrial fibrillation via a ventricular lead.

FIG. 8 is a flow chart illustrating an example method for detecting or confirming atrial fibrillation via a ventricular lead.

FIG. 9 is a flowchart illustrating an example method for detecting atrial fibrillation and storing information relating to the atrial fibrillation based on the detection.

### DETAILED DESCRIPTION

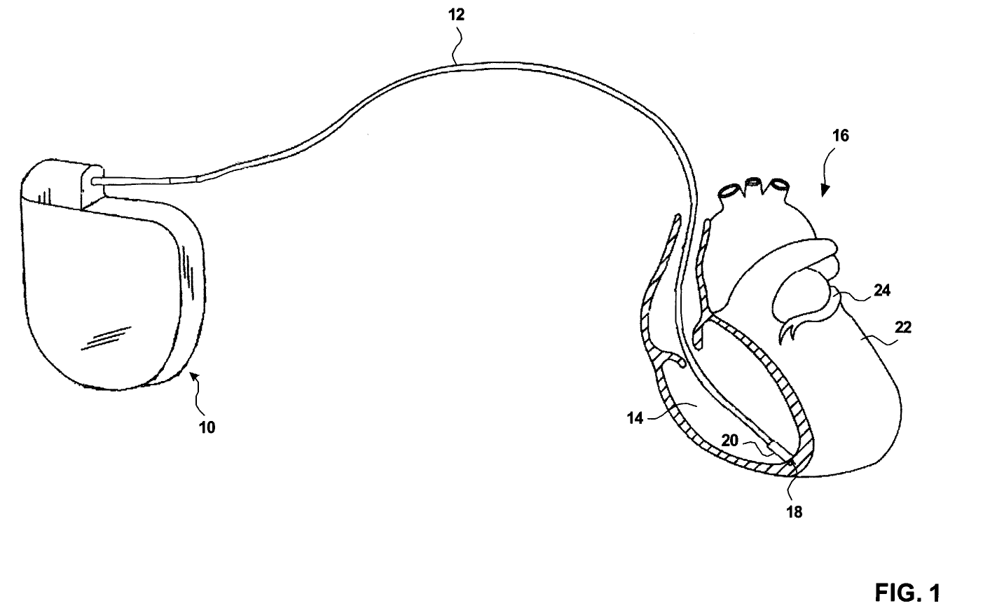

FIG. 1 is a perspective diagram illustrating an implantable medical device (“IMD”) 10 electrically coupled to a ventricular pacing and sensing lead 12. As will be described in greater detail below, IMD 10 detects atrial fibrillation via lead 12, and stores information relating to detected atrial fibrillations in a memory. IMD 10 may take the form of a cardiac pacemaker, pacemaker-cardioverter-defibrillator, implantable loop recorder, or the like.

By storing information relating to detected atrial fibrillations, IMD 10 may allow a physician to later review the information and diagnose atrial fibrillation in situations where atrial fibrillation may have otherwise been unsuspected and undetected. This, in turn, may allow the physician to prescribe proper therapy for atrial fibrillation, such as anticoagulation therapy to reduce the risk of stroke resulting from atrial fibrillation. The information stored by IMD 10 may include beginning and ending times for atrial fibrillation episodes, the duration of atrial fibrillation episodes, a number of atrial fibrillation episodes detected, and a total amount of time of atrial fibrillation episodes detected.

Lead 12 may, as shown in FIG. 1, extend from IMD 10 into a right ventricle 14 of a heart 16. A bipolar electrode pair 18,20 may be located proximate to a distal end of lead 12 within right ventricle 14. IMD 10 may monitor depolarizations of right ventricle 14 via electrodes 18,20, and may detect atrial fibrillation based on the monitored depolarizations. Further, in some embodiments, IMD 10 may deliver pacing pulses to right ventricle 14 via electrodes 18, 20, and detect atrial fibrillation by monitoring the response of right ventricle 14 to the delivered pacing pulses.

The configuration and location of lead 12 and electrodes 18, 20 is, however, merely exemplary. Lead 12 need not include bipolar electrode pair 18, 20, but may instead include a unipolar electrode that cooperates with an electrode located on the housing of IMD 10. Further, lead 12 may extend to any location within or proximate to either right ventricle 14 or a left ventricle 22 of heart 16. For example, lead 12 may be a coronary sinus lead that extends into a coronary sinus 24 of heart 16 to a position proximate to left ventricle 22.

Although IMD 10 may detect atrial fibrillation via a single ventricular lead 12, IMD 10 may in some embodiments detect atrial fibrillation via multiple ventricular leads. Further, IMD 10 may be coupled to any number of additional leads in order to provide pacing, cardioversion, defibrillation, or other therapies to heart 16. Nonetheless, the ability of IMD 10 to detect atrial fibrillation via a single ventricular lead 12 may provide advantages.

For example, detection of atrial fibrillation via a single ventricular lead 12 may allow a reduction in the complexity and cost of IMD 10, avoid the cost of additional leads 12, and allow for a reduction in the total amount of material implanted in a patient. Moreover, atrial fibrillation may be detected in situations where it would not have otherwise been detected, such as in situations where the previous condition of the patient had only indicated the need for a simple IMD 10, such as a single-lead pacemaker operating in VVI mode.

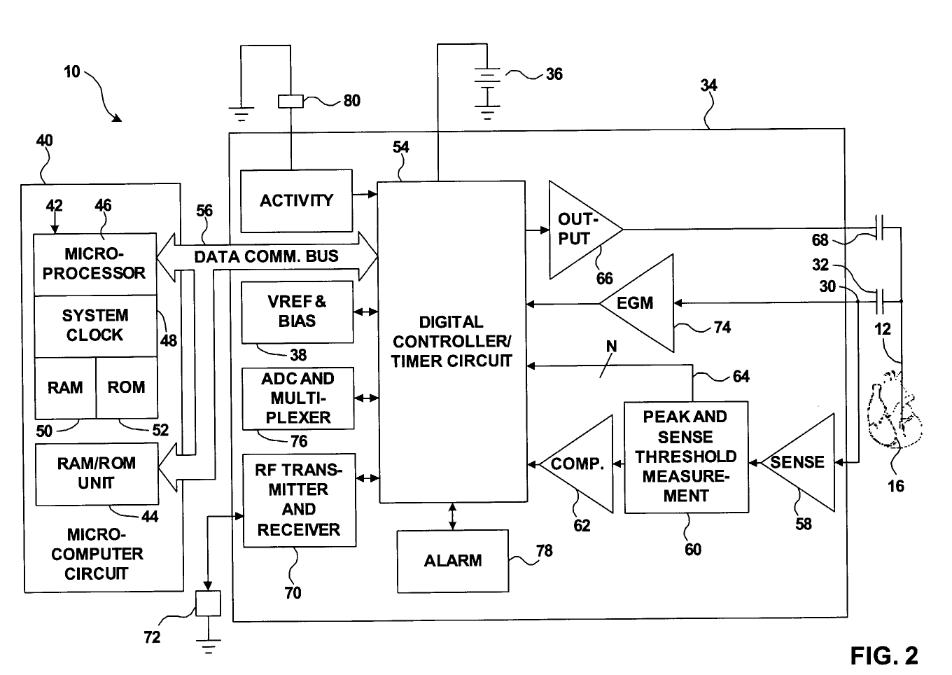

FIG. 2 is a block diagram further illustrating an exemplary embodiment of IMD 10. In particular, FIG. 2 illustrates constituent components of IMD 10 in accordance with one embodiment of the present invention, where IMD 10 is a pacemaker having a microprocessor-based architecture. As shown in FIG. 2, lead 12 is coupled to a node 30 in IMD 10 through an input capacitor 32. Input/output circuit 34 contains circuits for interfacing lead 12 and the various components of IMD 10. A battery 36 may provide power to the various components of IMD 10, and a VREF and bias circuit 38 may generate stable voltage reference and bias currents for the various components of IMD 10.

IMD 10 includes a processor and memory, such as microcomputer circuit 40 shown in FIG. 2. Microcomputer circuit 40 may include on-board circuit 42 and off-board circuit 44. On-board circuit 42 preferably includes microprocessor 46, system clock circuit 48 and on-board RAM 50 and ROM 52. Off-board circuit 44 preferably comprises a RAM/ROM unit. Microcomputer circuit 40 may comprise a custom integrated circuit device augmented by standard RAM/ROM components.

Microprocessor 46 may execute instructions stored in any computer-readable medium suitable for storing instructions including on-board RAM 50, ROM 52, off-board circuit 44, non-volatile random access memory (NVRAM) (not shown), electrically erasable programmable read-only memory (EEPROM) (not shown), flash memory (not shown), and the like. Some of these instructions cause microprocessor 46 to perform the functions attributed to microprocessor 46 herein. In particular, some of these instructions may cause microprocessor 46 to detect atrial fibrillation via ventricular lead 12, and store information relating to the detected atrial fibrillation as described herein.

Microprocessor 46 is coupled to a digital controller/timer circuit 54 via a data communications bus 56. Circuit 54 is in turn coupled to sensing circuitry, including sense amplifier 58, peak sense and threshold measurement unit 60, and comparator/threshold detector 62. Sense amplifier 58 amplifies sensed electrical cardiac signals and provides an amplified signal to peak sense and threshold measurement circuitry 60, which in turn provides an indication of peak sensed voltages and measured sense amplifier threshold voltages on multiple conductor signal path 64 to digital controller/timer circuit 54. An amplified sense amplifier signal is also provided to comparator/threshold detector 62.

Sense amplifier 58, peak sense and threshold measurement unit 60 and comparator/threshold detector 62 may be used in the practice of the present invention to detect the occurrence of ventricular depolarizations by detecting R-waves in the signal received from ventricular lead 12. Microprocessor 46 may receive signals indicating the occurrence of ventricular depolarizations via digital controller/timer circuit 54 and data communications bus 56, and may use the indications to detect atrial fibrillation. As will be described in greater detail below, microprocessor 46 may use the indications to evaluate the rate of ventricular depolarizations, and/or the stability of the rate of ventricular depolarizations, in order to detect atrial fibrillation via ventricular lead 12. Additionally or alternatively, as will be described in greater detail below, microprocessor 46 may control circuit 54 to cause output pulse generator 66 to provide a pacing pulse to ventricle 14 through coupling capacitor 68 and lead 12, and may detect the response of heart 16 to delivery of the pacing pulse in order to detect or confirm atrial fibrillation. Microprocessor 46 may measure R-R interval by measuring the time period between R-wave occurrence signals received from pacer timing and control circuit 54.

Microprocessor 46 stores information relating to detected atrial fibrillation episodes in a memory, such on on-board or off-board RAM units 50,44. As mentioned above, the information may include beginning and ending times for atrial fibrillation episodes, the duration of atrial fibrillation episodes, a number of atrial fibrillation episodes detected, and a total amount of time of atrial fibrillation episodes detected. A physician may later retrieve the stored information to diagnose atrial fibrillation for a patient in whom IMD 10 is implanted. Physicians may retrieve the information using a programmer or patient monitor (not shown) that is capable of communicating with microprocessor 46 via an RF transmitter and receiver 70 and antenna 72.

In some embodiments, physicians may also program aspects of the behavior of IMD 10, and receive other information stored by microprocessor 46 via a programmer. For example, the physician may receive a stored or real-time electrogram (EGM) signal via a programmer. Digital controller/timer circuit 54 may be coupled to an electrogram (EGM) amplifier 74 for receiving amplified and processed signals sensed by ventricular lead 12. An ADC and multiplexer unit 76 may digitize the EGM signal received by circuit 54, and the digitized EGM signal may be received by microprocessor 46 via data communications bus 56. As another example, the physician may retrieve information concerning the operation of IMD 10, such as information related to the level of charge remaining in battery 36.

IMD 10 may further include an alarm 78 that provides an audible signal to the patient in whom IMD 10 is implanted. Microprocessor 46 may activate alarm 78 in response to detected atrial fibrillation to alert the patient to a possible problem that may require consultation with a physician. Microprocessor 46 may not activate alarm 78 in response to all detected episodes of atrial fibrillation, but may instead activate alarm 78 when the detected episode satisfies a preprogrammed criterion. For example, microprocessor 46 may compare the duration of an episode of atrial fibrillation to a threshold value stored in one of RAM 50, ROM 52, or off-board RAM/ROM 44, and if the duration meets or exceeds the threshold value, may then activate alarm 78.

The invention is not limited to the microprocessor-based pacemaker embodiment of IMD 10 depicted in FIG. 2. In some embodiments, IMD 10 may not provide pacing therapy at all. Instead, IMD 10 may monitor and record physiological events. In embodiments where IMD 10 does provide pacing therapy, digital timers and counters of digital controller/timer circuit 54 establish the overall escape interval of the IMD 10 as well as various refractory, blanking and other timing windows for controlling the operation of peripheral components disposed within input/output circuit 34. The durations of these intervals are determined by microprocessor 46 in response to data stored in RAM 50, ROM 52, or off-board RAM/ROM 44, and/or received from a programmer via RF transmitter and receiver 70. The durations are then communicated to circuit 54 via data communications bus 56.

Depending on the number and configuration of leads associated with IMD 10, IMD 10 may provide DDD, VVI, DVI, VDD, DDI and other modes of single and dual chamber pacing well known to the art. Further, in various embodiments of the present invention, IMD 10 may be rate responsive. In such embodiments, digital controller/timer circuit 50 may vary the rate based on signals received from an activity sensor 80, which may include a piezoceramic accelerometer. Activity sensor 80 typically (although not necessarily) provides a sensor output that varies as a function of a measured parameter relating to the metabolic requirements of a patient.

Numerous pacemaker features and functions not explicitly mentioned herein may be incorporated into IMD 10 while remaining within the scope of the present invention. Moreover, as mentioned above, in some embodiments IMD 10 may take the form of a pacemaker-cardioverter-defibrillator. Such embodiments of IMD 10 may execute program instructions and include components known in the art for detecting arrhythmias, and for providing anti-tachycardia pacing therapies, cardioversion therapies, and defibrillation therapies.

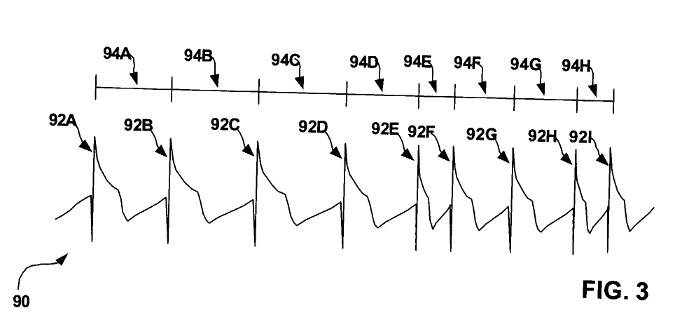

FIG. 3 is a timing diagram used to illustrate exemplary methods that may be employed by IMD 10 to detect atrial fibrillation via ventricular lead 12. In particular, FIG. 3 illustrates an example ventricular EGM signal 90. EGM signal 90 represents electrical activity within heart 16 that may be sensed by sensing circuitry 58-62 via electrodes 18,20 and lead 12.

EGM signal 90 includes R-waves 92A-I (collectively “R-waves 92”), which correspond to depolarizations of ventricles 14,22. As described above, microprocessor 46 of IMD 10 receives indications of the occurrence of R-waves 92 from digital controller/timing circuit 54, and thus receives indications of the occurrence of ventricular depolarizations. Based on these indications, microprocessor 46 measures the period of time between R-waves 92, i.e., R-R intervals 94A-H (collectively “R-R intervals 94”), and thus measures the amount of time between ventricular depolarizations.

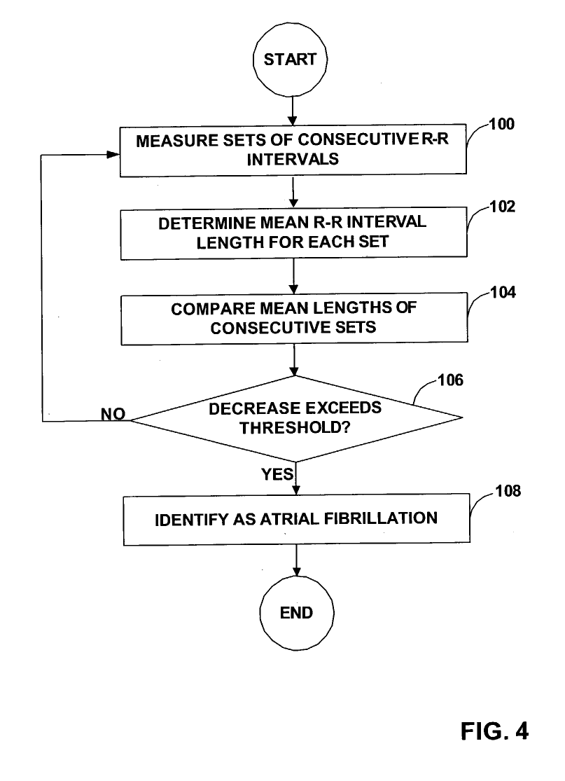

Microprocessor 46 may determine a rate of depolarizations of ventricles 14, 22, i.e., a ventricular rate, based on the length of R-R intervals 94. Microprocessor 46 may detect atrial fibrillation based on the ventricular rate. Microprocessor 46 may detect atrial fibrillation by, for example, detecting the rate of change of the ventricular rate, monitoring the stability of the ventricular rate, or monitoring the rate of change of the stability of the ventricular rate, as will be described below with reference to FIGS. 4-6.

FIG. 4 is a flowchart illustrating an example method that may be employed by IMD 10 to detect atrial fibrillation via ventricular lead 12. In particular, FIG. 4 illustrates an example method that may be employed by IMD 10 to detect atrial fibrillation by detecting the rate of change of the ventricular rate. As mentioned above, conducted atrial fibrillation causes an increase in the ventricular rate.

Microprocessor 46 of IMD 10 may measure R-R intervals 94, and group R-R intervals 94 into sets (100). For example, microprocessor 46 may group measured R-R intervals 94 into sets of four, e.g., a first set that includes intervals 94A-D and a second set that includes intervals 94E-H. Microprocessor 46 may determine the mean R-R interval length for each set (102), and compare the mean lengths of consecutive sets (104). If the decrease in mean length between adjacent sets exceeds a threshold value stored in a memory (106), such as RAM 50, ROM 52, or off-board RAM/ROM 44, microprocessor 46 may determine that atrial fibrillation is occurring (108). Detecting a decrease in the ventricular rate is particularly useful for detecting the onset of atrial fibrillation, which will cause an initial increase in the ventricular rate.

The use of sets including four R-R intervals is merely exemplary. Sets may include any number of R-R intervals, or the ventricular rate may be evaluated beat-to-beat. The use of sets, however, militates against potential erroneous detection of atrial fibrillation based on spurious beat-to-beat changes in the ventricular rate.

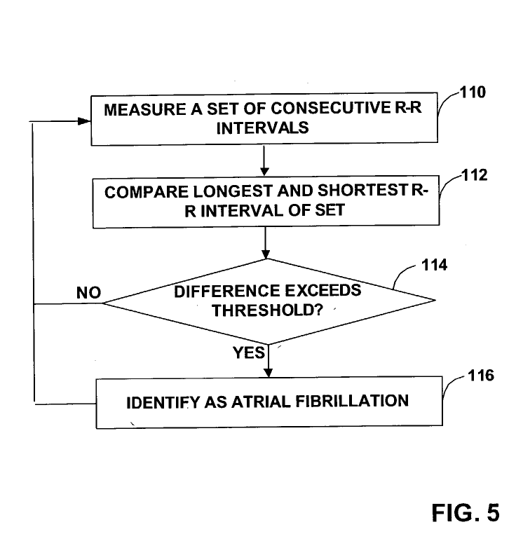

FIG. 5 is a flowchart illustrating another example method that may be employed by IMD 10 to detect atrial fibrillation via ventricular lead 12. In particular, FIG. 5 illustrates an example method that may be employed by IMD 10 to detect atrial fibrillation by monitoring the stability of the ventricular rate. As mentioned above, conducted atrial fibrillation may cause the ventricular rate to be less stable.

Microprocessor 46 of IMD 10 may measure a set of R-R intervals 94 (110). As described above, the set may contain any number of R-R intervals 94. For example, microprocessor 46 may group intervals 94 into sets of four, and a set might include intervals 94A-D.

Microprocessor 46 may then compare the longest and shortest interval 94 of the set (112), and determine if the difference between the longest and shortest interval exceeds a threshold value stored in a memory, such as RAM 50, ROM 52, or off-board RAM/ROM 44 (114). If the difference exceeds the threshold, microprocessor 46 may determine that atrial fibrillation is occurring (116). If not, microprocessor may evaluate the next set of intervals 94E-H.

The difference between the longest and shortest interval 94 in a set indicates the stability of the ventricular rate within the time period corresponding to the set. Thus, a threshold value and number of intervals per set should be chosen such that the stability threshold is appropriate to discern between instability caused by conducted atrial fibrillation, and normal ventricular rate instability. Further, the invention is not limited to use of the difference between the longest and shortest interval 94 of a set to monitor stability. Rather, various other statistical evaluations of the length of intervals 94 within a set may be made to determine stability. For example, the statistical variance, standard deviation, standard error, root mean squared difference, or the like of a set of intervals 94 may be calculated, and used to evaluate the stability of the ventricular rate.

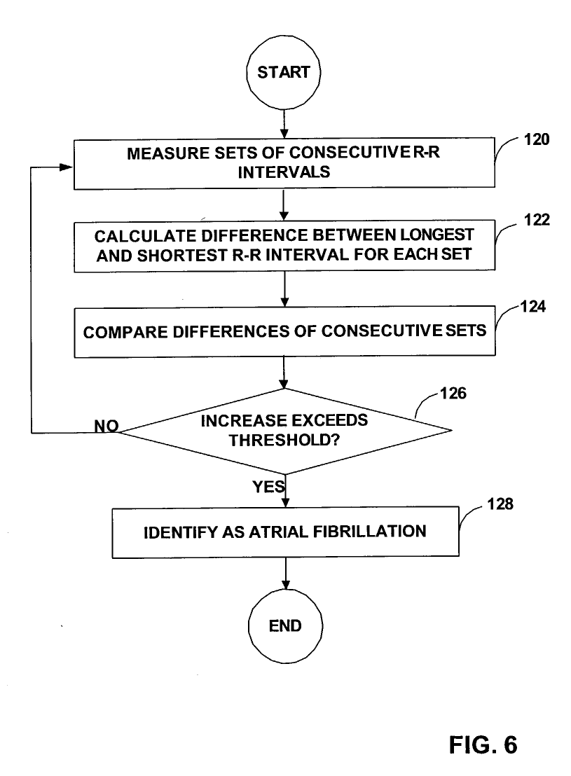

FIG. 6 is a flowchart illustrating another example method that may be employed by IMD 10 to detect atrial fibrillation via ventricular lead 12. In particular, FIG. 6 illustrates an example method that may be employed by IMD 10 to detect atrial fibrillation by monitoring the rate of change in the stability of the ventricular rate. By detecting a significant decrease in the stability of the ventricular rate, IMD 10 may be able to detect the onset of atrial fibrillation, which if conducted may cause the ventricular rate to become less stable.

Microprocessor 46 of IMD 10 may measure sets of consecutive R-R intervals (120), and calculate the difference between the longest and shortest R-R intervals for each set (122). Microprocessor 46 may compare the differences for consecutive sets (124), and determine whether an increase in the value of the difference between consecutive sets is greater than a threshold value stored in a memory, such as RAM 50, ROM 52, or off-board RAM/ROM 44 (126). If the increase in the value exceeds the threshold, microprocessor 46 may determine that atrial fibrillation is occurring (128). Again, any measurement of the stability of the ventricular rate may be used.

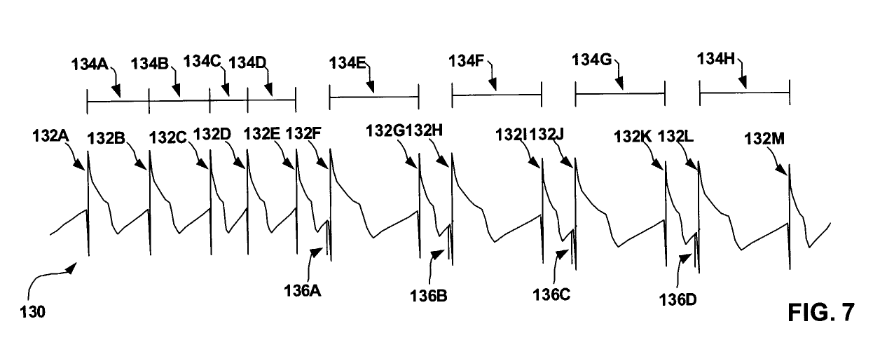

FIG. 7 is a timing diagram used to illustrate an exemplary method that may be employed by IMD 10 to detect or confirm atrial fibrillation via ventricular lead 12. In particular, FIG. 7 illustrates an example ventricular EGM signal 130 that includes R-waves 132A-M (collectively “R-waves 132”). FIG. 7 further illustrates R-R intervals 134A-H (collectively “R-R intervals 134”) that may be measured by microprocessor 46 based on detected R-waves 132.

As mentioned above, IMD 10 may deliver pacing pulses, such as pacing pulses 136A-D (collectively “pulses 136”) illustrated in FIG. 7, to ventricle 14 through lead 12, and may detect the response of heart 16 to delivery of pulses 136 in order to detect or confirm atrial fibrillation. During an episode of conducted atrial fibrillation, a pulse 136 that captures ventricles 14,22 will lead to a detectably longer R-R interval 134 subsequent to delivery of the 136. This longer R-R interval 136 may be referred to as a compensatory pause, and indicates concealed retrograde conduction to the A-V node of heart 16, which occurs during episodes of conducted atrial fibrillation. Thus, for example, microprocessor 46 may detect atrial fibrillation by measuring intervals 134E-H, which occur subsequent to pulses 136A-D, to determine if they reflect a compensatory pause.

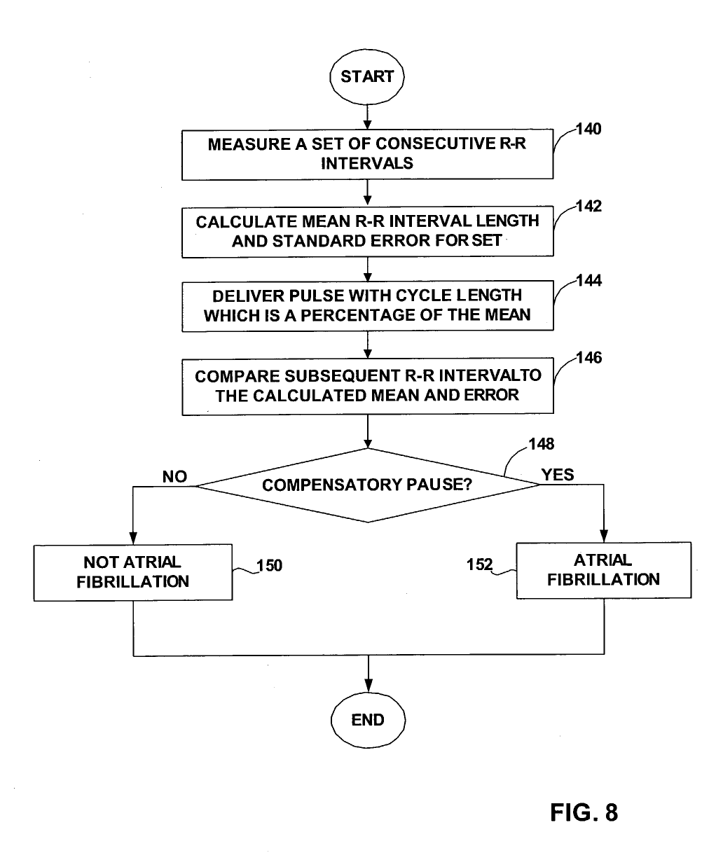

FIG. 8 is a flowchart illustrating an example method that may be employed by IMD 10 to detect or confirm atrial fibrillation based on the detection of compensatory pauses. Microprocessor 46 may measure a set of R-R intervals 134 (140), such as R-R intervals 134A-D, and calculate the mean R-R interval length and standard error for the set (142). Again the set may include any number of intervals 134.

Microprocessor 46 then controls delivery of pacing pulses 136 (144). In order to promote capture of ventricles 14,22, microprocessor 46 may control delivery pulses 136 with a cycle length that is a fraction or percentage of the calculated mean. After each of pulses 136A-D is delivered, microprocessor 46 may measure the subsequent R-R intervals 134E-H to determine if the length of the interval indicates a compensatory pause (146-152). One way that microprocessor 46 may make this determination is to compare the subsequent interval 134 to the calculated mean. For example, microprocessor 46 may compare the subsequent interval 134 to the sum of the mean and a value determined by multiplying the calculated standard error with a constant stored in a memory, such as RAM 50, ROM 52, or off-board RAM/ROM 44.

If a compensatory pause is indicated, microprocessor 46 may determine that atrial fibrillation is occurring. In some embodiments, microprocessor 46 may consider the response of heart 16 to several pulses 138 before making the determination. For example, microprocessor 46 may make the determination based on the fraction of a total number of pacing pulses that result in a compensatory pause. Further, the mean and standard error may be calculated, or the fraction or percentage of the mean may be adjusted a number of times before microprocessor 46 makes the determination. In some embodiments, microprocessor 46 may use the detection of compensatory pauses to confirm the existence of atrial fibrillation after it has been detected by one of the other method discussed above with reference to FIGS. 3-6.

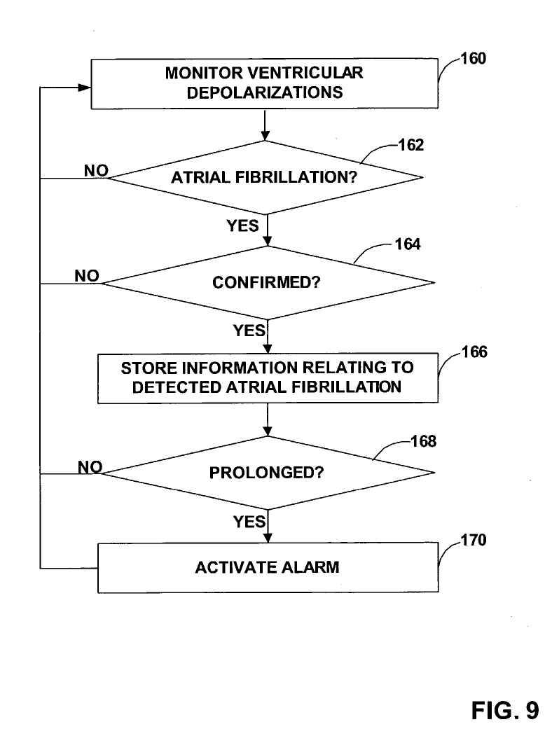

FIG. 9 is a flowchart illustrating an example method that may be employed by IMD 10 to detect atrial fibrillation and store information relating to the atrial fibrillation based on the detection. Microprocessor 46 monitors ventricular depolarizations as described above (160), and uses any of the above-described methods to detect atrial fibrillation (162). In some embodiments, microprocessor 46 may employ more than one or all of the method described above. For example, microprocessor may detect an increase in the ventricular rate and/or instability to detect an atrial fibrillation episode, and monitor the stability of ventricular rate to determine the duration and ending time of the episode. In some embodiments, microprocessor 46 may confirm the detected atrial fibrillation (164) by, for example, detecting the occurrence of compensatory pauses in response to delivery of pacing pulses 136, as described above.

When atrial fibrillation is detected, or, in embodiments that include confirmation, confirmed, microprocessor 46 may store information relating to the detected atrial fibrillation episode in a memory, such as RAM 50, ROM 52, or off-board RAM/ROM 44 (166). As mentioned above, the information may include beginning and ending times for atrial fibrillation episodes, the duration of atrial fibrillation episodes, a number of atrial fibrillation episodes detected, and a total amount of time of atrial fibrillation episodes detected. A physician may later retrieve the stored information to diagnose atrial fibrillation for a patient in whom IMD 10 is implanted.

Further, microprocessor 46 may activate an alarm 78 in response to the detected atrial fibrillation (170). In some embodiments, microprocessor 46 may activate the alarm in response to a determination that the detected atrial fibrillation episode is prolonged (168).

Various embodiments of the invention have been described. However, one skilled in the art will appreciate that various modifications may be made to the described embodiments without departing from the claimed invention. For example, although described herein with reference a to microprocessor-based pacemaker, IMDs according to the invention are not so limited. IMDs according to the invention may include a processor that may be implemented as an embedded microprocessor, a controller, a DSP, an ASIC, an FPGA, discrete logic circuitry, or the like. These and other embodiments are within the scope of the following claims.

What is claimed is:

1. A method comprising:
detecting atrial fibrillation via a ventricular lead; and
storing information relating to the detected atrial fibrillation in a memory.

2. The method of claim 1, wherein detecting atrial fibrillation comprises:
monitoring stability of a ventricular rate via the lead; and
detecting atrial fibrillation based on the stability of the rate.

3. The method of claim 2, wherein monitoring stability comprises:
measuring a set of adjacent R-R intervals;
comparing a longest R-R interval and a shortest R-R interval of the set; and
determining the stability of the ventricular rate based on the comparison.

4. The method of claim 1, wherein detecting atrial fibrillation comprises:
monitoring a rate of change in stability of a ventricular rate via the lead; and
detecting atrial fibrillation based on the rate of change in stability.

5. The method of claim 4, wherein monitoring a rate of change in stability comprises:
measuring sets of adjacent R-R intervals;
comparing a longest R-R interval and a shortest R-R interval for each set to determine a stability of the rate for each set; and
comparing the stability of adjacent sets.

6. The method of claim 1, wherein detecting atrial fibrillation comprises detecting an increase in a ventricular rate.

7. The method of claim 6, wherein detecting an increase in a ventricular rate comprises:
measuring sets of adjacent R-R intervals;
calculating a mean R-R interval length for each set; and
comparing the mean R-R interval lengths of adjacent sets.

8. The method of claim 1, wherein detecting atrial fibrillation comprises detecting a compensatory pause after a depolarization resulting from delivery of a pacing pulse via the ventricular lead.

9. The method of claim 8, further comprising:
measuring a set of R-R interval lengths; and
calculating a mean R-R interval length for the set, wherein detecting a compensatory pause comprises delivering a pacing pulse with a cycle length that is a fraction of the mean, and comparing a length of an R-R interval that occurs subsequent to delivery of the pulse to the mean.

10. The method of claim 1, further comprising confirming the detected atrial fibrillation, wherein storing information comprises storing information based on the confirmation.

11. The method of claim 10, wherein confirming the detected atrial fibrillation comprises detecting a compensatory pause after a depolarization resulting from delivery of a pacing pulse via the ventricular lead.

12. The method of claim 1, wherein the information includes at least one of a time when the atrial fibrillation begins, a time when the atrial fibrillation ends, the duration of the atrial fibrillation, a number of atrial fibrillation episodes detected, and a total amount of time of atrial fibrillation episodes detected.

13. The method of claim 1, further comprising activating an alarm based on the detection.

14. The method of claim 13, wherein activating an alarm comprises:
determining a duration of the detected atrial fibrillation; and
activating the alarm based on the duration.

15. An implantable medical device comprising:
a memory to store information; and
a processor to detect atrial fibrillation via a ventricular lead, and store information relating to the detected atrial fibrillation in the memory.

16. The implantable medical device of claim 15, wherein the processor monitors stability of a ventricular rate via the lead, and detects atrial fibrillation based on the stability of the rate.

17. The implantable medical device of claim 16, wherein the processor measures a set of adjacent R-R intervals, compares a longest R-R interval and a shortest R-R interval of the set, and determines the stability of the ventricular rate based on the comparison.

18. The implantable medical device of claim 15, wherein the processor monitors a rate of change in stability of the ventricular rate, and detects atrial fibrillation based on the rate of change in stability.

19. The implantable medical device of claim 18, wherein the processor monitors the rate of change in stability by measuring sets of adjacent R-R intervals, comparing a longest R-R interval and a shortest R-R interval for each set to determine the stability of the rate for each set, and comparing the stability of adjacent sets.

20. The implantable medical device of claim 15, wherein the processor detects atrial fibrillation based on an increase in a ventricular rate.

21. The implantable medical device of claim 20, wherein the processor detects atrial fibrillation by measuring sets of adjacent R-R intervals, calculating a mean R-R interval for each set, and comparing the mean R-R intervals of adjacent sets.

22. The implantable medical device of claim 15, wherein the processor detects atrial fibrillation by detecting a compensatory pause after a depolarization resulting from delivery of a pacing pulse via the ventricular lead.

23. The implantable medical device of claim 22, wherein the processor measures a set of R-R interval lengths, calculates a mean R-R interval length for the set, controls delivery of a pacing pulse with a cycle length that is a fraction of the mean, and compares a length of an R-R interval that occurs subsequent to delivery of the pulse to the mean.

24. The implantable medical device of claim 15, wherein the processor confirms the detected atrial defibrillation, and stores the information based on the confirmation.

25. The implantable medical device of claim 24, wherein the processor confirms the detected atrial defibrillation by detecting a compensatory pause after a depolarization resulting from delivery of a pacing pulse via the ventricular lead.

26. The implantable medical device of claim 15, wherein the information includes at least one of a time when the atrial fibrillation begins, a time when the atrial fibrillation ends, the duration of the atrial fibrillation, a number of atrial fibrillation episodes detected, and a total amount of time of atrial fibrillation episodes detected.

27. The implantable medical device of claim 15, further comprising an alarm, wherein the processor activates the alarm based on the detection.

28. The implantable medical device of claim 27, wherein the processor determines a duration of the detected atrial fibrillation, and activates the alarm based on the duration.

29. The implantable medical device of claim 15, wherein a distal end of the lead includes an electrode, and the electrode is located in a right ventricle.

30. A computer-readable medium comprising instructions that cause a processor to:
detect atrial fibrillation via a ventricular lead; and
store information relating to the detected atrial fibrillation in a memory.

31. The computer-readable medium of claim 30, wherein the instructions cause a processor to:
monitor stability of a ventricular rate via the lead; and
detect atrial fibrillation based on the stability of the rate.

32. The computer-readable medium of claim 31, wherein the instructions cause a processor to:
measure a set of adjacent R-R intervals;
compare a longest R-R interval and a shortest R-R interval of the set; and
determine the stability of the ventricular rate based on the comparison.

33. The computer-readable medium of claim 30, wherein the instructions cause a processor to:
monitor a rate of change in stability of the ventricular rate; and
detect atrial fibrillation based on the rate of change in stability.

34. The computer-readable medium of claim 33, wherein the instructions that cause a processor to monitor a rate of change in stability comprise instructions that cause a processor to:
measure sets of adjacent R-R intervals;
compare a longest R-R interval and a shortest R-R interval for each set to determine the stability of the rate for each set; and
compare the stability of adjacent sets.

35. The computer-readable medium of claim 30, wherein the instructions cause a processor to detect atrial fibrillation based on an increase in a ventricular rate.

36. The computer-readable medium of claim 35, wherein the instructions that cause a processor to detect an increase in a ventricular rate comprise instructions that cause a processor to:
measure sets of adjacent R-R intervals;
calculate a mean R-R interval length for each set; and
compare the mean R-R interval lengths of adjacent sets.

37. The computer-readable medium of claim 30, wherein the instructions that cause a processor to detect atrial fibrillation comprise instructions that cause a processor to detect a compensatory pause after a depolarization resulting from delivery of a pacing pulse via the ventricular lead.

38. The computer-readable medium of claim 37, further comprising instructions that cause a processor to:
measure a set of R-R interval lengths; and
calculate a mean R-R interval length for the set, wherein the instructions that cause a processor to detect a compensatory pause comprise instructions that cause a processor to control delivery of a pacing pulse with a cycle length that is a fraction of the mean, and compare a length of an R-R interval that occurs subsequent to delivery of the pulse to the mean.

39. The computer-readable medium of claim 30, further comprising instructions that cause a processor to confirm the detected atrial fibrillation, wherein the instructions cause a processor to store information based on the confirmation.

40. The computer-readable medium of claim 39, wherein the instructions that cause a processor to confirm the detected atrial fibrillation comprise instructions that cause a processor to detect a compensatory pause after a depolarization resulting from delivery of a pacing pulse via the ventricular lead.

41. The computer-readable medium of claim 30, wherein the instructions cause a processor to store at least one of a time when the atrial fibrillation begins, a time when the atrial fibrillation ends, the duration of the atrial fibrillation, a number of atrial fibrillation episodes detected, and a total amount of time of atrial fibrillation episodes detected.

42. The computer-readable medium of claim 30, further comprising instructions that cause a processor to activate an alarm based on the detection.

43. The computer-readable medium of claim 42, wherein the instructions cause a processor to:
determine a duration of the detected atrial fibrillation; and
activate the alarm based on the duration.

44. An implantable medical device comprising:
means for detecting atrial fibrillation via a ventricular lead; and
means for storing information relating to the detected atrial fibrillation in a memory.

45. The implantable medical device of claim 44, wherein the means for detecting atrial fibrillation comprises:
means for monitoring stability of a ventricular rate via the lead; and
means for detecting atrial fibrillation based on the stability of the rate.

46. The implantable medical device of claim 45, wherein the means for monitoring stability comprises:
means for measuring a set of adjacent R-R intervals;
means for comparing a longest R-R interval and a shortest R-R interval of the set; and
means for determining the stability of the ventricular rate based on the comparison.

47. The implantable medical device of claim 44, wherein the means for detecting atrial fibrillation comprises:
means for monitoring a rate of change in stability of a ventricular rate via the lead; and
means for detecting atrial fibrillation based on the rate of change in stability.

48. The implantable medical device of claim 44, wherein the means for detecting atrial fibrillation comprises means for detecting an increase in a ventricular rate.

49. The implantable medical device of claim 44, wherein the means for detecting atrial fibrillation comprises means for detecting a compensatory pause after a depolarization resulting from delivery of a pacing pulse via the ventricular lead.

50. The implantable medical device of claim 44, further comprising means for confirming the detected atrial fibrillation, wherein the means for storing information stores information based on the confirmation.

51. The implantable medical device of claim 44, further comprising means for activating an alarm based on the detection.
